(19) United States
(12) Patent Application Publication
(10) Pub. No.: US 2014/0251388 A1
(43) Pub. Date: Sep. 11, 2014

(54) CLEANING SYSTEM AND METHOD OF CLEANING

(71) Applicant: BIYOUNG BIOTECHNOLOGY CO., LTD., Tortola (GB)

(72) Inventors: Chien-An CHEN, Kaohsiung (TW); SHIH-MING TUNG, Kaohsiung (TW); YEN-HUI CHEN, Kaohsiung (TW)

(73) Assignee: BIYOUNG BIOTECHNOLOGY CO., LTD., Tortola (GB)

(21) Appl. No.: 13/845,756

(22) Filed: Mar. 18, 2013

(30) Foreign Application Priority Data
Mar. 8, 2013 (TW) 102108326

(51) Int. Cl. A61C 17/02; A61C 17/20; B08B 3/10

(52) U.S. Cl. CPC A61C 17/0205; B08B 3/10; A61C 17/0202; A61C 17/20
USPC 134/36; 134/102.1; 134/57 R; 433/88; 433/82

(57) ABSTRACT

A method of supplying oxygenated water is performed on a water tank, in which oxygenated water is received. The method lets the water tank supply a constant amount of the oxygenated water once, and then supply the water tank with water and pure oxygen to recover the dissolution ratio of oxygen of the oxygenated water in the water tank. The method may supply a constant amount of oxygenated water every time for the user to drink it up once.

### BACKGROUND OF THE INVENTION

1. Technical Field

The present invention relates to cleaning equipment, and more particularly to a cleaning system and a method of cleaning.

2. Description of Related Art

In present days, people live in a highly pathenogenic environment because of frequent contact with other people and appalling sanitation. Therefore, it is more and more important to keep a clean public environment and a good personal hygiene.

In various pathenogenic bacteria, anaerobic bacteria are highly concerned in the medical field. More than 50% surgical infections are caused by anaerobic bacteria. Anaerobic bacteria lead to serious thoracic/abdominal infections and abscesses, and lead to soft tissue necrosis as well. Typically, there are five groups of anaerobic bacteria, including 1). Bacteroides fragilis; 2). Prevotella and Porphyromonas; 3). F. nucleatum; 4). Peptostreptococcus; and 5). C. perfringenes and C. ramosum. The anaerobic bacteria live in lots of organs, such as oral cavity, nasal cavity, throat, and nose.

In oral cavity, more than 50% anaerobic bacteria live in salive, and most of them live in tartar and periodontal pocket. Toothbrush is the commonest tool to clean teeth and oral cavity. However, there still are anaerobic bacteria remained in oral cavity after brushing teeth, and it will lead to halitosis and calculus. After a long time, people have to see dentist for calculus removal. Ultrasonic scaler is the commonest equipment for dentist to remove calculus. The ultrasonic scaler provides an ultrasonic water jet for the dentist to remove calculus with a hand piece. The conventional ultrasonic scaler only provides ultrasonic water jet to scale calculus off. However, it can't remove the anaerobic bacteria in the same time, so that calculus will grow in a short time, and people have to see the dentist frequently.

### BRIEF SUMMARY OF THE INVENTION

In view of the above, the primary objective of the present invention is to provide a cleaning system and a method of cleaning, which provides oxygenated water for cleaning and disinfecting at the same time. It is effective for a medical purpose, especially in inhibition of anaerobic bacteria.

The present invention provides a cleaning system, including an oxygen supplier, an exchanging unit, and an oxygenated water provider. The oxygen supplier provides pure oxygen to the exchanging unit. The exchanging unit receives water from a water source and the pure oxygen from the oxygen supplier to dissolve the pure oxygen into the water in a first pressure so as to obtain oxygenated water. The oxygenated water provider receives the oxygenated water from the exchanging unit and releases the oxygenated water in a second pressure.

The present invention further provides a method of cleaning an object, including the following steps:

(a). Dissolve pure oxygen into water in a first pressure to obtain oxygenated water; and

(b) Release the oxygenated water in a second pressure to clean an object.

The present invention is applied in environment cleaning, medical purpose, or healthcare for calculus removal, prevention of anaerobic bacteria's growth, and sterilization.

### BRIEF DESCRIPTION OF THE SEVERAL VIEWS OF THE DRAWINGS

The present invention will be best understood by referring to the following detailed description of some illustrative embodiments in conjunction with the accompanying drawings, in which

FIG. 1 is a block diagram of the cleaning system of a first preferred embodiment of the present invention;

FIG. 2 is a block diagram of the cleaning system of a second preferred embodiment of the present invention;

FIG. 3 is a block diagram of the cleaning system of a third preferred embodiment of the present invention; and

FIG. 4 is a flowchart of the present invention.

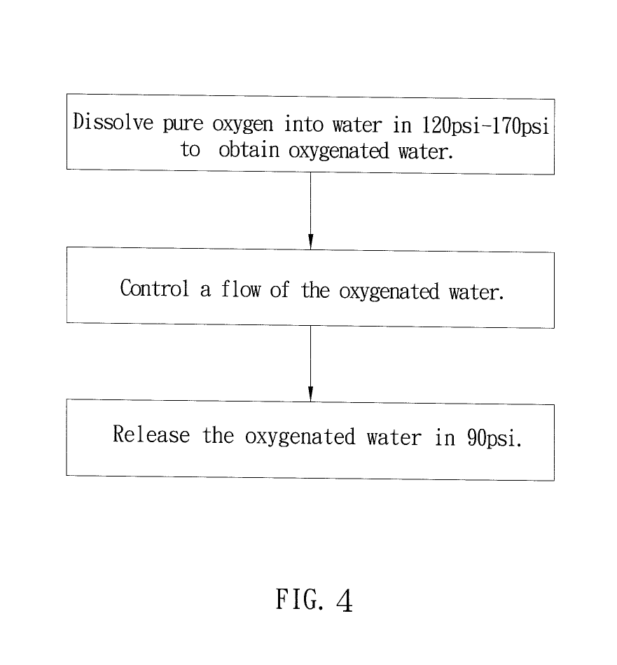

### DETAILED DESCRIPTION OF THE INVENTION

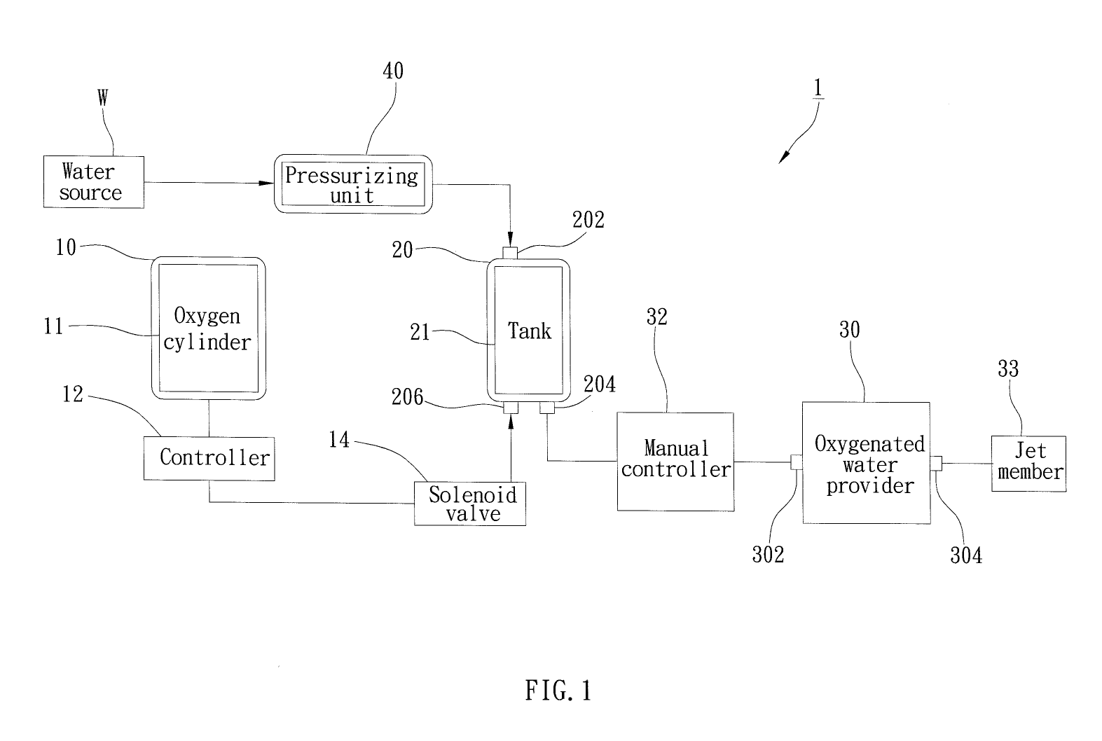

FIG. 1 shows a cleaning system 1 of the first preferred embodiment of the present invention, which includes an oxygen supplier 10, an exchanging unit 20, an oxygenated water provider 30, and a pressurizing unit 40, which is a motor in the present embodiment.

The oxygen supplier 10 has an oxygen cylinder 11 to supply pure oxygen. It may have two or more oxygen cylinders according to the requirement.

The exchanging unit 20 has a tank 21. The tank 21 has a first water inlet 202, a first water outlet 204, and a first gas inlet 206. The first water inlet 202 is on a top of the tank 21, and the first water outlet 204 and the first gas inlet 206 are on a bottom. A water source W is connected to the first water inlet 202 to supply the tank 21 with water. The pressurizing unit 40 pressurizes the water from the water source W before it flows into the tank 21. The water of the water source W preferable is drinking water, such as water from a reverse osmosis (RO) system or sterile water. The oxygen supplier 10 has a controller 12 and a solenoid valve 14. The oxygen supplier 10 is connected to the first gas inlet 206 of the tank 21, and a user may control the controller 12 and the solenoid valve 14 to adjust an oxygen flow to the tank 21. The water from the water source W falls down via the first water inlet 202, and the pure oxygen of the oxygen supplier 10 goes up via the first gas inlet 206, so that they will impact and mix in the tank 21. Furthermore, we keep a pressure in the tank 21 in a first pressure, between 120 psi and 170 psi, that would speed up the process of mixing and dissolution to obtain 100 ppm or higher oxygenated water.

The oxygenated water provider 30 has a second water inlet 302, a second water outlet 304, and a manual switch 32. A jet member 33, which is a nozzle in the present embodiment, is connected to the second water outlet 304 of the oxygenated water provider 30. The second water inlet 302 is connected to the first water inlet 204 of the exchanging unit 20 through a pipe. The manual switch 32 is on the pipe to turn on and turn off the oxygenated water. The oxygenated water comes out via the jet member 33 in a second pressure. In an embodiment, the second pressure is 90 psi, and the jet member 33 provides an oxygenated water jet to clean environment, prevent bacteria's growth, or clean oral cavity for medical purpose.

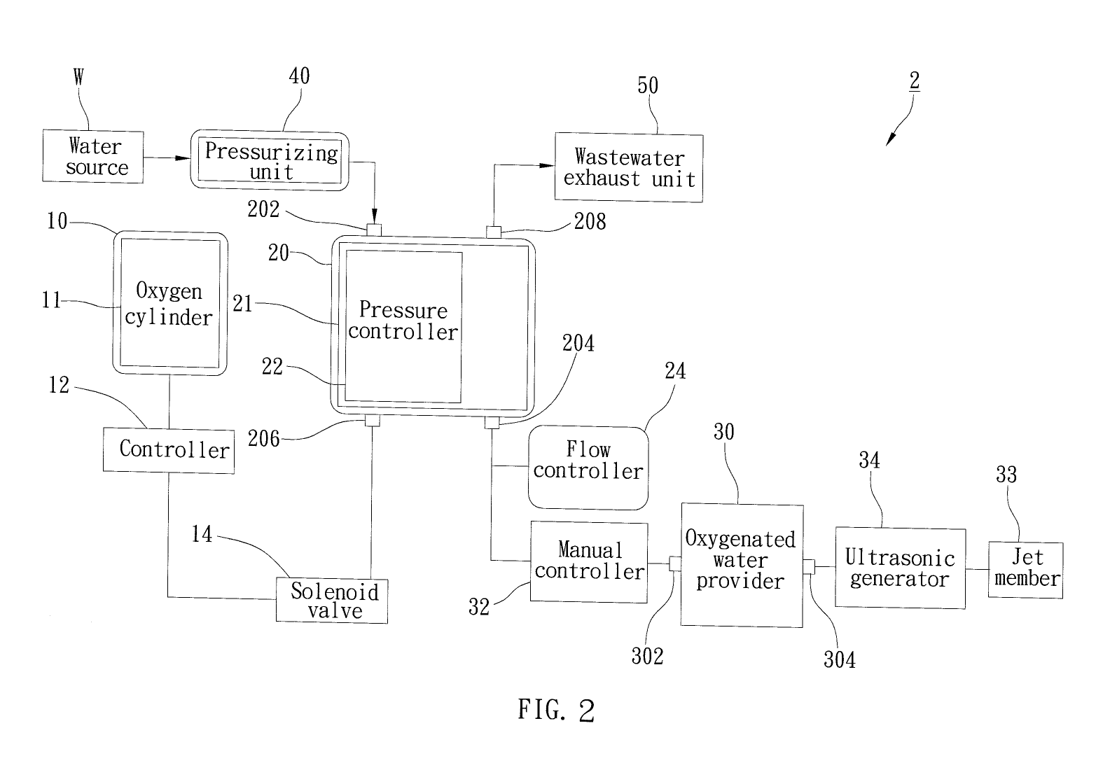

FIG. 2 shows a cleaning system 2 of the second preferred embodiment of the present invention. Basically, the cleaning system 2 of the second preferred embodiment is the same as the cleaning system 1 of the first preferred embodiment, except that

The cleaning system 2 further has an ultrasonic generator 34 between the oxygenated water provider 30 and the jet member 33. The ultrasonic generator 34 provides ultrasonic weaves in the oxygenated water, so that the cleaning system 2 may be used to remove calculus. In addition, the exchanging unit 20 further has a pressure controller 22 and a flow controller 24. The pressure controller 22 is in the tank 21 to sense a pressure in the tank 21. If the pressure controller 22 found a low pressure in the tank 21 (lower than the first pressure) the pressure controller 22 starts the pressurizing unit 40 to pressurize water from the water source W that would recover the pressure in the tank 21. The flower controller 24 is on a pipe, which is connected to the first water outlet 204 of the tank 21 to sense a volume of the oxygenated water coming out from the exchanging unit 20. When a specific volume is found, the flower controller 24 activates the controller 12 and the solenoid valve 14 to provide pure oxygen into the tank 21. The tank 21 further has a water outlet 208. The water outlet 208 connects to a wastewater exhaust unit 50 to exhaust wastewater of the exchanging unit 20.

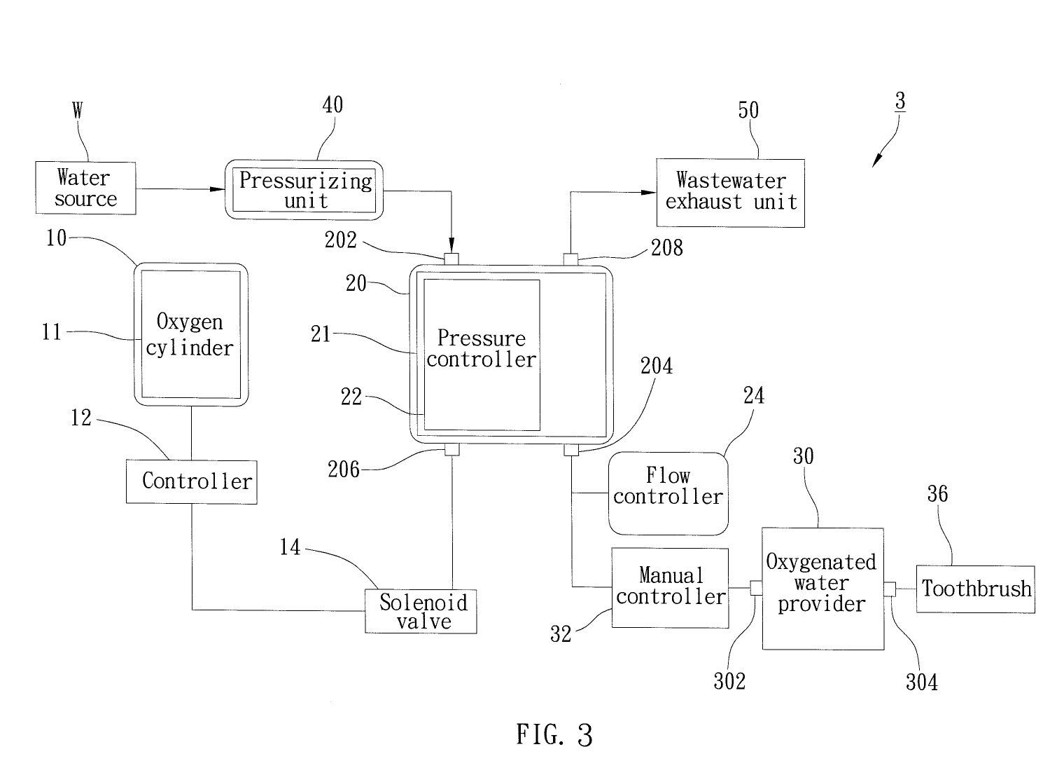

FIG. 3 shows a cleaning system 3 of the third preferred embodiment of the present invention. Basically, the cleaning system 3 of the third preferred embodiment is the same as the cleaning system 2 of the second preferred embodiment, except that the jet member 33 is a toothbrush 36 for a user to clean his/her oral cavity with oxygenated water when he/she brushes teeth.

A method of cleaning an object by the cleaning system 1, 2, or 3 includes the following steps:

(a). Pressurize water from the water source W by the pressurizing unit 40, supply the pressurized water to the tank 21 through the first water inlet 202, supply pure oxygen to the tank 21 through the first gas inlet 206, and keep a pressure in the tank between 120 psi and 170 psi (the first pressure). The pure oxygen is dissolved in the pressurized water in the tank 21 to obtain oxygenated water (100 ppm or higher).

(b). Release the oxygenated water in the tank 21 in a second pressure (90 psi) to clean an object. The cleaning method of the present invention may remove calculus, prevent anaerobic bacteria's growth, and sterilize.

The advantages of the present invention are:

1. The present invention may be applied in various fields of cleaning or sterilization, especially for medical purposes or healthcare, to reduce the risk of infection.

2. The present invention may be applied in dentistry field to replace the conventional ultrasonic scaler, and to reduce the usage of antibiotics.

3. The present invention provides the water coming down from top and pure oxygen going up from bottom that will speed up the mixture in the tank and obtain high oxygenated water. The whole size of the cleaning system is small enough for usage at home.

It must be pointed out that the embodiments described above are only some preferred embodiments of the present invention. All equivalent structures and manufacturing methods which employ the concepts disclosed in this specification and the appended claims should fall within the scope of the present invention.

What is claimed is:

1. A cleaning system, comprising:
an oxygen supplier for providing pure oxygen;
an exchanging unit receiving water from a water source and the pure oxygen from the oxygen supplier to dissolve the pure oxygen into the water in a first pressure so as to obtain oxygenated water; and
an oxygenated water provider connected to the exchanging unit, wherein the oxygenated water provider receives the oxygenated water from the exchanging unit and releases the oxygenated water in a second pressure.

2. The cleaning system of claim 1, further comprising a pressurizing unit between the water source and the exchanging unit to pressurize the water from the water source and keep the exchanging unit in the first pressure.

3. The cleaning system of claim 2, wherein the exchanging unit has a tank, and the tank has a first water inlet, a first water outlet, and a first gas inlet; the oxygenated water provider has a second water inlet and a second water outlet; the water source is connected to the first water inlet of the tank through the pressurizing unit; the oxygen supplier is connected to the first gas inlet of the tank; and the second water inlet of the oxygenated water provider is connected to the first water outlet of the tank.

4. The cleaning system of claim 3, wherein the oxygen supplier has a controller to adjust the pure oxygen supplied to the exchanging unit.

5. The cleaning system of claim 4, wherein the oxygen supplier has a solenoid valve between the controller and the exchanging unit, so that the oxygen supplier is connected to the first gas inlet of the tank through the controller and the solenoid valve.

6. The cleaning system of claim 5, wherein the oxygenated water provider has a jet member to release the oxygenated water in the second pressure.

7. The cleaning system of claim 6, wherein the oxygenated water provider further has a manual controller to turn on and turn off the oxygenated water from the exchanging unit to the oxygenated water provider.

8. The cleaning system of claim 7, wherein the exchanging unit further has a water outlet, and the water outlet is connected to a wastewater exhaust unit to exhaust wastewater in the exchanging unit.

9. The cleaning system of claim 2, wherein the exchanging unit has a pressure controller to sense a pressure in the exchanging unit and adjust the pressure to the first pressure.

10. The cleaning system of claim 2, wherein the exchanging unit has a flow controller to sense a quantity of the oxygenated water coming out from the exchanging unit and control the oxygen supplier to provide pure oxygen into the exchanging unit.

11. The cleaning system of claim 2, wherein the first pressure is between 120 psi and 170 psi; the second pressure is 90 psi, and a concentration of the oxygenated water is higher than 100 ppm.

12. The cleaning system of claim 6, wherein the jet member is a nozzle or a toothbrush.

13. The cleaning system of claim 9, wherein the jet member is a nozzle or a toothbrush.

14. The cleaning system of claim 9, further comprising an ultrasonic generator between the oxygenated water provider and the jet member to provide ultrasonic weaves in the oxygenated water.

15. A method of cleaning an object, comprising the steps of:
(a) dissolving pure oxygen into water in a first pressure to obtain oxygenated water; and
(b) releasing the oxygenated water in a second pressure to clean an object.

16. The method of claim 15, wherein the first pressure is between 120 psi and 170 psi.

17. The method of claim 15, wherein the second pressure is 90 psi.

18. The method of claim 15, wherein the step (a) is performed in a tank; and the water enters the tank from a top thereof, and the pure oxygen enters the tank from a bottom thereof.

19. The method of claim 15, wherein the method is applied in environment cleaning, medical purpose, or healthcare.
